(19) United States
(12) Patent Application Publication
(10) Pub. No.: US 2017/0175278 A1
(43) Pub. Date: Jun. 22, 2017

(54) Oxygen Gas Supply Device and Method

(71) Applicant: Reactive Innovations, LLC, Westford, MA (US)

(72) Inventors: Michael C. Kimble, Westford, MA (US); Daniel Carr, Leominster, MA (US)

(21) Appl. No.: 14/977,904

(22) Filed: Dec. 22, 2015

(51) Int. Cl. C25B 9/10; A61M 16/08; A61M 16/10; B01D 53/32; C25B 1/04

(52) U.S. Cl. CPC C25B 9/10; B01D 53/326; C25B 1/04; A61M 16/1005; A61M 16/0875; B01D 2053/223

(57) ABSTRACT

An oxygen gas supply device includes a tubular hydrated ion-exchange membrane defining an inner surface, an outer surface and an outlet. An outer catalytic membrane at the outer surface and an inner catalytic membrane at the inner surface are in electrical communication with a direct current power source. Application of electromotive force between the outer and inner catalytic membranes causes an oxygen gas component of the ambient air in contact with one or the other of the outer and inner catalytic membranes to be separated and collected at the other catalytic membrane and thereby be collected as an oxygen gas supply.

### BACKGROUND

Supplemental oxygen delivery systems are vital to provide a critical life-support respiratory function for patients suffering from lung diseases, and for other users needing an independent oxygen supply for various purposes, such as occupational or safety purposes. For example, supplemental oxygen is necessary for patients suffering from lung diseases, including: pulmonary fibrosis and sarcoidosis, as well as other ailments that weaken the respiratory system, such as heart disease and autoimmune deficiency disease. Over six million people in the United States alone are affected by chronic obstructive pulmonary disease (COPD) where oxygen therapy is prescribed.

Oxygen delivery devices and methods typically include or employ pressurized oxygen tanks or absorption-based oxygen concentrators. Portable oxygen concentrators often employ elevated temperatures and pressures to extract oxygen from ambient air. Modalities for delivering oxygen to ambulatory patients usually include high-pressure gas cylinders and oxygen concentrators based on pressure-swing absorption or hollow-fiber membranes. However, such devices generally are heavy, consume power at a high rate, emit large amounts of waste heat and are relatively noisy, thereby significantly limiting patient utility.

Therefore, a need exists to overcome or minimize the above-referenced problems.

### SUMMARY OF THE INVENTION

The invention generally is directed to an oxygen gas supply device and method that electrochemically separates oxygen from air by chemically reacting a hydrogen ion (a proton) with oxygen gas in the ambient air to form water, transports the water through an ion exchange membrane via diffusion, and subsequently oxidizes the water to form oxygen gas.

In one embodiment, the oxygen gas supply device includes a tubular hydrated ion-exchange membrane defining an inner surface, an outer surface and a port. An outer catalytic membrane is at the outer surface of the ion-exchange membrane, while an inner catalytic membrane is at the inner surface. The inner catalytic membrane has an inner surface that defines, at least in part, an inner tubular volume that is in fluid communication with the a port. A direct current power source is in electrical communication with the outer and inner catalytic membranes, whereby either the outer catalytic membrane or the inner catalytic membrane operates as a cathode, and the other of the outer catalytic membrane and the inner catalytic membrane operates as an anode. A manifold is in fluid communication with the port, whereby application of electromotive force across the tubular hydrated ion-exchange membrane by the direct current power source will cause a cathodic reaction of an oxygen gas component of ambient air at the cathode with a hydrogen ion to form water, and an anodic reaction of water at the anode to react to form oxygen gas, thereby causing oxygen gas to collect either within the inner tubular volume and pass through the port to the manifold as an oxygen gas supply, or at the outer catalytic membrane as an oxygen gas supply.

In another embodiment, the oxygen gas supply device includes a tubular hydrated ion-exchange membrane defining an inner surface, an outer surface and a port. An outer catalytic membrane is at the outer surface of the tubular hydrated ion-exchange membrane, and an inner catalytic membrane is at the inner surface of the tubular hydrated ion-exchange membrane, wherein the inner catalytic membrane with a hydrogen ion has an inner surface that defines, at least in part, an inner tubular volume that is in fluid communication within the port. A manifold is in fluid communication with the outlet, whereby application of electromotive force across the outer and inner catalytic membranes will cause a cathodic reaction of an oxygen gas component of ambient air at either the outer catalytic membrane or the inner catalytic membrane with a hydrogen ion to form water and an anodic reaction of water at the other of the outer catalytic membrane and inner catalytic membrane to form oxygen gas, thereby causing oxygen gas to collect either within the inner tubular volume and pass through the port to the manifold as an oxygen gas supply, or at the outer catalytic membrane for collection as the oxygen gas supply.

In still another embodiment, the invention is directed to a method for separating oxygen gas from air, including the step of exposing a tubular membrane to the ambient air, the tubular membrane including an outer catalytic membrane, an inner catalytic membrane within the outer catalytic membrane, and a tubular hydrated ion-exchange membrane contacting and partitioning the outer and inner catalytic membranes, the tubular membrane including an outlet in fluid communication with an inner tubular volume defined, at least in part, by an inner surface of the inner catalytic membrane. An electromotive force is applied between the outer and inner catalytic membranes, whereby an oxygen gas component of the ambient air will react in a cathodic reaction at either the outer catalytic membrane or the inner catalytic membrane with a hydrogen ion to form water, and water at the other of the outer catalytic membrane and the inner catalytic membrane will react in an anodic reaction to form oxygen gas that collects as an oxygen gas supply.

In yet another embodiment, the invention is directed to a portable wearable oxygen gas supply device. The portable wearable oxygen gas supply device includes a housing defining a first port and a second port, and is configured to be worn by a subject. A plurality of tubular membranes is located within the housing. Each of the tubular membranes includes a tubular hydrated ion-exchange membrane that defines an inner surface and an outer surface. An outer catalytic membrane is at the outer surface and in fluid communication with the first port, and an inner catalytic membrane is at the inner surface that is in fluid communication with the second port, wherein the inner catalytic membrane has an inner surface that defines an inner tubular volume. The portable oxygen gas supply device also includes terminals for a connection of the outer and inner catalytic membranes to a direct current power source, and a manifold includes communication with the either the first or the second port, whereby application of electromotive force across the tubular hydrated ion-exchange membranes by a direct current from a power source will cause a catalytic reaction of an oxygen component of ambient air at either the outer catalytic membranes or the inner catalytic membranes with a hydrogen ion to form water and an anodic reaction of water at the other of the outer catalytic membranes and the inner catalytic membranes to form oxygen gas, thereby causing oxygen gas to collect either within the inner tubular volumes and pass through the first port or the second port, separate from the ambient air to thereby form a supply of oxygen gas. The portable wearable oxygen supply device also includes a conduit extending from either first port or the second port of the housing, whereby the subject can access the supply of oxygen gas.

This invention has many advantages. For example, the oxygen supply device and method of the invention can produce up to six liters per minute of pulsed oxygen supply delivered to the patient on each inhalation, or up to one liter per minute of continuous oxygen supply to the patient packaged in a compact and portable device. The electrochemical separation process enables the device to operate noiselessly, a major benefit toward improving the quality of life of respiratory patients. Furthermore, the device operates using the inherent hydration state of the ion-exchange membranes eliminating the need to supply supplement water to the device. Thus, the device needs a replaceable or rechargeable battery and the ambient air to operate. This oxygen supply device is lightweight and compact that can be worn, for example, on the user's hip or in a shoulder sling. Therefore, the device and method of the invention provide improved mobility and, consequently, enhanced quality of life. Further, the oxygen supply device and method of the invention has other uses, such as an emergency oxygen supply for various medical applications beyond chronic disease, and for use where occupational or safety concerns are implicated, such as firefighting, exposure to toxic chemicals and space exploration.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a perspective view of one embodiment of a tubular membrane oxygen gas supply device of the invention.

FIG. 2A is a schematic representation of another embodiment of a tubular membrane oxygen gas supply device of the invention.

FIG. 2B is a cross-sectional view of the tubular membrane oxygen gas supply device of the invention of FIG. 2A taken along line 2B-2B.

FIG. 3 is a schematic representation of an embodiment of an oxygen gas supply device of the invention including a plurality of tubular membranes.

FIG. 4 is a cross-sectional representation of one embodiment of a plurality of tubular membranes of the portable wearable oxygen gas supply device of the invention.

FIG. 5 is another embodiment of a cross-sectional representation of a plurality of tubular membranes of the portable wearable oxygen gas supply device of the invention.

FIG. 6 is another embodiment of a cross-sectional representation of a plurality of tubular membranes of the portable wearable oxygen gas supply device of the invention.

FIG. 7 is another embodiment of a cross-sectional representation of a plurality of tubular membranes of the portable wearable oxygen gas supply device of the invention.

### DETAILED DESCRIPTION OF THE INVENTION

A description of example embodiments of the invention follows.

The invention generally is directed to an electrochemical separation device and method that chemically reacts the oxygen with hydrogen in ambient air to form a water molecule, transports the water molecule through an ion exchange membrane via diffusion, and subsequently oxidizes the water back to oxygen. Specifically, an electromotive force is supplied across an ion exchange membrane whereby, on a cathode side of the ion exchange membrane, air is introduced to a cell under ambient conditions, where oxygen is reduced by reaction with a hydrogen ion (a proton) to form water as shown in Eq. [1]. This water is transported across the ion exchange membrane via diffusion to an anode on the opposite side of the ion exchange membrane, where it is oxidized to form oxygen, thereby releasing a proton, as shown in Eq. [2]. This proton migrates through the membrane back to the cathode where it reacts with incoming oxygen of ambient air to produce water, thereby completing a cycle within the membrane. The net reaction is shown by Eq. [3] that shows oxygen being separated from air to form a stream of oxygen gas. During generation of the oxygen gas stream, water is conserved in the ion exchange membrane, meaning that no other reactants are required for the method and device of the system to function. Rather, only ambient air and a source of direct current are required to operate the device and method of the invention.

Cathode: O₂+N₂+4H⁺+4e⁻=2H₂O(1)+N₂E°=1.23V Eq. [1]

Anode: 2H₂O(1)=O₂+4H⁺+4e⁻E°=−1.23V Eq. [2]

Net: O₂(g)=O₂(g)E°=0.0V Eq. [3]

Although the cell potential across the ion exchange membrane, according to the device and method of the invention, can be as low as zero volts, typically a higher potential of about 1.5 volts is employed to drive the separation process and yet not reduce protons to hydrogen gas at the cathode.

In a specific embodiment, the invention employs micro-tubular electrochemical cells that are combined together to form modular units. Inside each tubular cell is an anode, while a cathode is on the outer surface of each tubular cell. In this manner, a collection of tubular cells is employed to contact ambient air at outer surfaces of the tubular cells. Oxygen in the ambient air reacts at the cathode to form water that diffuses to the anode at the interior surface of the tubular cells. Oxidizing this water liberates oxygen gas into the interiors of the tubular cells, which are connected to a common manifold to collect the separated oxygen gas.

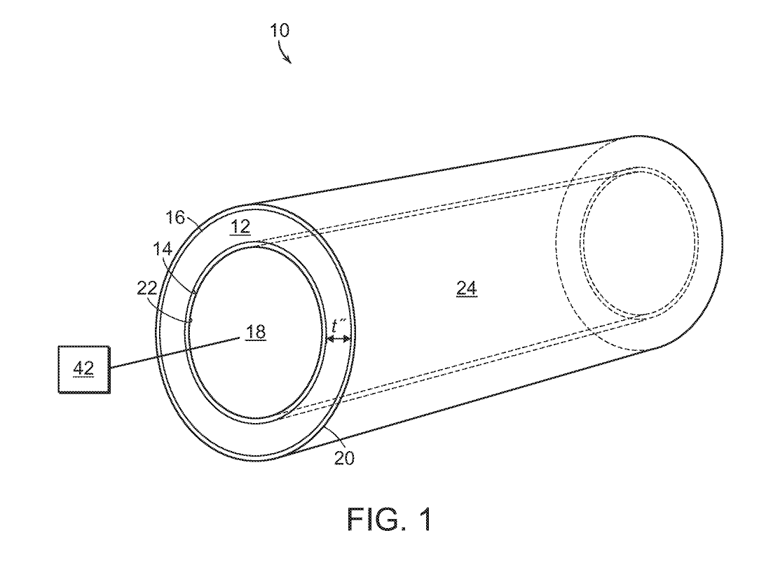

In one specific embodiment of an oxygen gas supply device of the invention, shown in FIG. 1, tubular membrane 10 includes hydrated ion-exchange membrane separator 12 that defines inner surface 14, outer surface 16 and outlet 18. Examples of suitable hydrated ion-exchange membranes separators 12 include hydrated ion-exchange membranes of at least one material selected from the group consisting of perfluorosulfonic acid (Nafion™), polysulfone, or other ion-exchange membrane materials. The term “hydrated ion-exchange membrane,” as employed herein, means an ion-exchange membrane that includes water in an amount in a range of between about 0 and about 40%. Preferably, the amount of water in the hydrated ion-exchange membrane is in a range of between about 1 and about 10%. Most preferably, the amount of water present in the hydrated ion-exchange membrane is between about 3 and 7%.

The hydrated ion-exchange membrane is prepared by a suitable method, such as by a method known in the art for hydrating ion-exchange materials. Generally, hydration is achieved by twice boiling the membrane in a 0.5 M sulfuric acid solution for one hour, followed by boiling the membrane in distilled water, twice, for one hour each.

In a particular preferred embodiment, the hydrated ion-exchange membrane is perfluorsulfonic acid NAFION® that is prepared by an extrusion process. An ion-exchange membrane is prepared by a melt extrusion process using precursor polymer pellets. Size and shape of polymeric membrane is dependent on extruder nozzle size. Shapes can include: circle, rectangle, square, triangle, star and other 3 dimensional shapes. Membranes can also be fabricated using a hot press method or dip coating. The polymeric membrane is then hydrolyzed using a multi-step chemical hydrolysis procedure to make the membrane ionically conductive. Once hydrolyzed, the ion-exchange membrane can be hydrated.

The unhydrolyzed polymeric membrane is fabricated into a tubular form by a suitable method, such as by extrusion. The polymeric membrane is formed via an extrusion process before it undergoes various chemical treatment steps to get the membrane in the ion exchange form. These steps, called hydrolysis, giving the membrane its ion exchange properties allowing the membrane to transport ions as well as take in water and become hydrated. Once the membrane is in the ion exchange form the membrane is hydrated. Typically hydrated ion-exchange membrane has a thickness, “t,” in a range of between about 0.001 inches and about 0.020 inches. Preferably, the range is between about 0.002 inches and about 0.004 inches.

Tubular membrane 10 further includes outer catalytic membrane 20 at outer surface 16 and inner catalytic membrane 22 at inner surface 14. Inner catalytic membrane 22 defines inner volume 24 that is in fluid communication with outlet 18. Examples of suitable materials of outer catalytic membrane 20 and inner catalytic membrane 22 include platinum, iridium oxide and ruthenium oxide. Inner catalytic membrane 22 and outer catalytic membrane 20 are each independently selected from the group consisting of platinum, iridium oxide and ruthenium oxide. Outer catalytic membrane 20 and inner catalytic membrane 22 are fabricated by a suitable technique known in the art. Inner and exterior catalytic membrane surfaces fabricated from the group consisting of electroless plating, electrolytic plating, sputtering and decal lamination. Typically, each of outer catalytic membrane 20 and inner catalytic membrane 22 has a thickness in a range of between about 0.0001 inches and about 0.5000 inches. Preferably, the thickness of each of inner catalytic membrane 22 and outer catalytic membrane 20 is in a range of between about 0.0001 inches and about 0.025 inches. In a particularly preferred embodiment, the thickness of outer catalytic membrane 20 is in a range between about 0.0001 inches and about 0.005 inches. In a particularly preferred embodiment, the thickness of the inner catalytic membrane 22 is in a range between a 0.0001 inches of 0.001 inches. The thicknesses of outer catalytic membrane 20 and inner catalytic membrane 22 can be the same as each other, or different. Preferably, the ratio of thickness of outer catalytic membrane 20 to that of inner catalytic membrane 22 is in a range of about 1:1 to about 1:10.

In one embodiment, wherein tubular hydrated polymeric membrane 10 has a circular cross-section, inner tubular volume 24 has a diameter in a range of between about 0.010 inches and about 0.499 inches.

Tubular hydrated ion-exchange membrane 10 has a suitable cross-sectional shape, such as a shape that is selected from the group consisting of a circle and a polygon. In the embodiment wherein tubular hydrated ion-exchange membrane 10 has a cross-sectional shape that is a polygon, the shape can be selected, for example, from the group consisting of a triangle, a rectangle and a square.

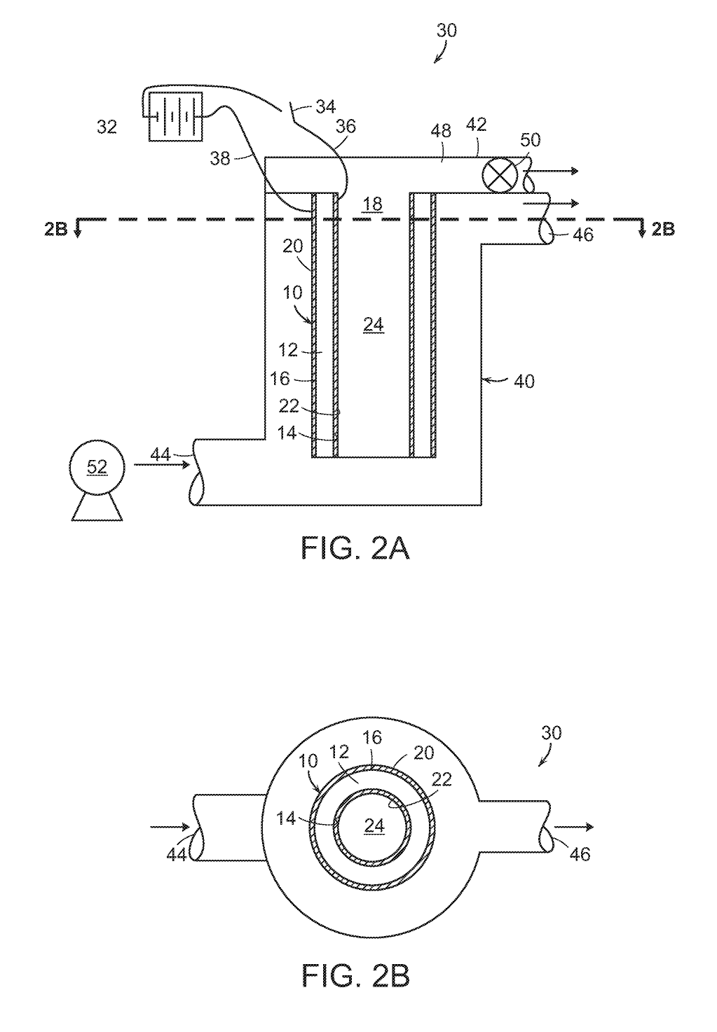

An embodiment of an oxygen gas supply device 30 of the invention, shown in FIGS. 2A and 2B, includes tubular hydrated ion-exchange membrane 10 having membrane separator 12 defining inner surface 14, outer surface 16 and outlet 18. Outer catalytic membrane 20 is at outer surface 16, while inner catalytic membrane 22 is at inner surface 14. Inner catalytic membrane 22 has an inner surface that defines, at least in part, inner tubular volume 24 that is in fluid communication with outlet 18. Direct current power source 32 is in electrical communication and, in one embodiment, selective electrical communication via switch 34, with outer catalytic membrane 20 and inner catalytic membrane 22, whereby outer catalytic membrane 20 operates as a cathode, and inner catalytic membrane 22 operates as an anode. Typically, direct current power source is a battery, as shown in FIG. 2A. The term “selective,” as that term is employed herein, means that electrical communication can be manually or automatically turned on, off, or otherwise adjusted or changed. In one embodiment, battery 32 provides an electromotive force sufficient to cause a cathodic reaction of an oxygen gas component of ambient air at outer catalytic membrane 20 to react to form water by reacting with protons available in tubular hydrated polymeric membrane separator 12, and to cause an anodic reaction of water at inner catalytic membrane 22 to react to form oxygen gas and protons. Although not shown, the polarity of the inner and outer catalytic membranes can be reversed. In one embodiment, the electromotive force applied by direct current power source 32 is in a range of between about 0.0 and about 1.5 volts. Preferably, the electromotive force applied by the direct current power supply is between 0.10 and 1 volts. Most preferably, the electromotive force applied via the direct power supply is between 0.50 and 0.75 volts.

Generally, inner current collector 36 extends between inner catalytic membrane 22 and the positive terminal of direct current power source 32, while outer current collector 38 extends between the outer catalytic membrane 20 and the negative terminal of direct current power source 32, thereby providing electrical communication between direct current power source 32, outer catalytic membrane 20 and inner catalytic membrane 22. Current collectors 36, 38 are each formed of suitable materials, such as those known in the art. Examples of suitable materials that can be employed to fabricate current collectors include platinum clad copper, carbon fiber, platinum and titanium. In one embodiment, current collectors are each independently selected from the group consisting of wires, grades, meshes, porous frits, foams, woven mats, pads, porous tubes, porous particles, slotted tubes, and slotted rods. In one particular embodiment, current collector 36 is formed inside tubular hydrated ion-exchange membrane separator 12 during fabrication of tubular hydrated ion-exchange membrane 10, such as during an extrusion process to fabricate tubular hydrated polymeric membrane separator 12. In another embodiment, current collector 38 is formed at outer catalytic membrane 20 by a suitable method, such as by a method known in the art. Examples of suitable methods include fabrication by at least one method selected from the group consisting of wrapping, braiding or placement at an outer surface of outer catalytic membrane 20.

Housing 40 defines manifold 42 that is in fluid communication with outlet 18, whereby oxygen gas generated within tubular 24 volume passes through outlet 18 and manifold 48 as an oxygen gas supply. Housing 40 also defines ambient air inlet 44 and ambient air outlet 46.

It is to be understood throughout that, in an alternative embodiment, the polarity of the electrodes can be reversed, as well as the roles of the inlet 44 and outlet 46, whereby outer catalytic membrane 20 operates as an anode, inner catalytic membrane 22 operates as a cathode, air outlet 46 operates as an ambient air inlet, and ambient air inlet 44 operates as an air outlet.

In one embodiment, a surface area-to-volume ratio, as defined by an outer surface of outer catalytic membrane 20 relative to inner tubular volume 24 is equal to or greater than about 16:1 in²/in³. In another embodiment, tubular hydrated ion-exchange membrane 10 has an axial length in a range of between about 0.125 inches and about 24.0 inches. In a specific embodiment, manifold 42 includes conduit 48 and regulator valve 50, whereby the supply of oxygen gas from conduit 48 through regulator valve 50 to a patient is regulated. Air pump 52 can be employed to direct ambient air from inlet 44 to outlet 46 of housing 40.

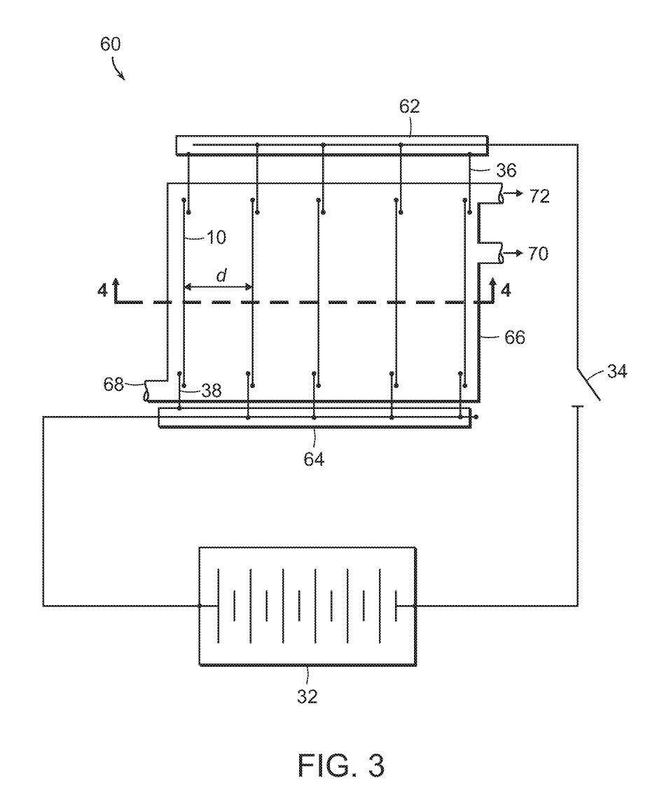

In another embodiment, shown schematically in FIG. 3, oxygen gas supply device 60 includes a plurality of tubular hydrated ion-exchange membranes, each of which includes corresponding inner catalytic membranes and outer catalytic membranes, as described above. Inner tubular volumes are each defined by inner surfaces of the inner catalytic membranes and are in fluid communication with manifold. Electrical terminals are in selective electrical communication with inner catalytic membranes and outer catalytic membranes to direct electrical current from a power source, as described above.

Current collectors 36, 38 provide electrical communication between direct current power source 32, inner catalytic membranes and outer catalytic membranes, respectively. First bus bar 62 electrically connects inner current collectors 36 extending from the inner catalytic membranes. Although not shown, the polarity of the inner catalytic membrane and the outer catalytic membranes can be reversed. In other words, in one embodiment the outer catalytic membrane can be anodic and the inner catalytic membrane can be cathodic, while in another embodiment the outer catalytic membrane can be cathodic and the inner catalytic membrane can be anodic. Second bus bar 64 connects outer current collectors 38 extending from the outer catalytic membranes. Inner current collectors 36 and outer current collectors 38 are secured by a suitable technique, such as is known in the art, including, for example, use of an electrically conductive epoxy, paint, solder, braze, spot-welding, crimping, compression fit or set screw. First bus bar 62 and second bus bar 64 can be formed of materials suitable for use with the present invention and can, in one embodiment be at least one material selected from the group consisting of copper, aluminum, carbon, platinum clad copper or some other suitable electrical conductor.

In one embodiment, inner current collectors 36 and first bus bar 62 are encapsulated, not shown, such as by use of an epoxy, to thereby enable first bus bar 62 to be removed from inner current collectors 36 and subsequently connect to another electrical connection or power supply. Examples of suitable bus bars include wires, braids, bars and rods, and can be either hung or solid for conducting electrical current to current collectors.

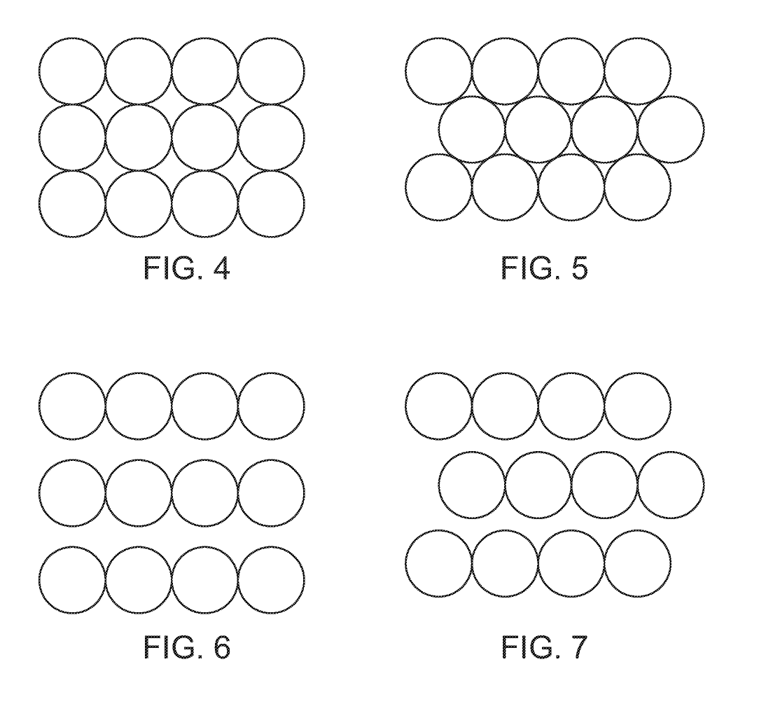

In one embodiment, outside surfaces of tubular hydrated ion-exchange membranes 10 can be separated from each other by a suitable distance, “d,” such as a distance in a range of between about 0.001 inches about 0.500 inches. In one particular embodiment, the distance between respective hydrated ion-exchange membranes 10 is about 0.25 inches. Although hydrated ion-exchange membranes in FIG. 3 are shown to be arranged in parallel, other arrangements are possible, such as offset or staggered arrangements, shown in FIGS. 4-7, which are different arrangements of cross-sections of tubular membrane 10, taken along line 4-4 of FIG. 3.

In one embodiment, a regulator is employed to restrict the flow of oxygen from outlet, thereby causing oxygen gas within inner volume and manifold to become pressurized as oxygenated gas is generated by application of electromotive force according to the method of the invention, as described above with reference to FIGS. 2A and 2B.

The embodiment of oxygen gas supply device 60 includes housing 66, defining ambient air inlet 68, ambient air outlet 70 and manifold 72, as described with reference to FIGS. 2A and 2B. Oxygen gas supply generated by the apparatus and method of the invention is directed from the apparatus of the invention through manifold 72.

The method of the invention includes exposing outer catalytic membrane 20 of FIG. 2, or the outer catalytic membrane 20 of FIG. 3, to ambient air. An electromotive force is applied by direct current power supply between the outer and inner catalytic membranes, whereby an oxygen gas component of ambient air will react in a cathodic reaction at outer catalytic membrane with a hydrogen ion (a proton) to form water, and water at inner catalytic membrane will react in an anodic reaction to form oxygen gas that will collect within the inner volume of each respective inner catalytic membrane. Oxygen gas generated at inner catalytic membrane is collected through an outlet of tubular membrane, thereby generating a supply of oxygen gas. In an alternative embodiment, the electrodes are reversed and air is directed inside the tubular cells, while oxygen is generated on the outer surface. This alternative mode of operation enables oxygen pressures to be generated such as up to or greater than about 500 pounds-force/square inch (“PSI”) (gage).

Air at ambient temperature and pressure is directed into the oxygen concentrator device using diffusion, convection, or other driving forces. Ambient temperatures include the range from 0° C. to 50° C. Ambient pressure includes those pressures from sea-level to altitudes greater than 30,000 ft. Nominal oxygen generation rates are 6.8 cc O₂/(cm²-hr) fed by an ambient air flow 5× greater, or 34.2 cc air/(cm²-hr). Ambient air diffusion to the tubular cells is sufficient eliminating active flow components including fans, blowers, and compressors enabling noiseless oxygen gas delivery.

Operation of the tubular cell is permissible using the inherent hydration state of the hydrated ion-exchange membrane where the membrane water content is used on the anode to be oxidized to produce oxygen gas, releasing a proton, and where this proton is subsequently reduced on the cathode with ambient oxygen in air, thereby reforming water to maintain the membrane's water content. Such operation eliminates the need to supply external water to operate the oxygen gas supply device. Higher oxygen gas supply delivery rates may be obtained by supplying extra water to the oxygen gas delivery system and operating the system at electromotive forces, such as electromotive forces up to or greater than about 1.5 V.

While this invention has been shown and described with reference to preferred embodiments thereof, it will be understood by those skilled in the art that various changes in form and detail may be made therein without departing from the scope of the invention encompassed by the appended claims.

1. A tubular membrane oxygen gas supply device, comprising:
a) a tubular hydrated ion-exchange separator defining an inner surface, an outer surface and a port;
b) an outer catalytic membrane at the outer surface;
c) an inner catalytic membrane at the inner surface, wherein the inner catalytic membrane has an inner surface that defines, at least in part, an inner tubular volume that is in fluid communication with the port;
d) a direct current power source in electrical communication with the outer and inner catalytic membranes, whereby one of the catalytic membranes operates as a cathode, and the other of the catalytic membranes operates as an anode; and
e) a manifold in fluid communication with the port, whereby application of an electromotive force across the tubular hydrated ion-exchange membrane by the direct current power source will cause a cathodic reaction of an oxygen gas component of ambient air at the cathode with hydrogen ions to form water, and an anodic reaction of water at the anode to react to form oxygen gas and hydrogen ions, thereby causing oxygen gas to collect either within the inner tubular volume and pass through the port to the manifold as an oxygen gas supply, or at the outer catalytic membrane as the oxygen gas supply.

2. The tubular membrane oxygen gas supply device of claim 1, wherein the outer catalytic membrane is a cathode, the inner catalytic membrane is an anode, and the oxygen gas collects within the inner tubular volume and passes through the port as an oxygen gas supply.

3. The oxygen gas supply device of claim 1, wherein the outer catalytic membrane is an anode and the outer catalytic membrane is a cathode, and the oxygen gas collects at the outer surface of the tubular hydrated ion exchange separator as the oxygen gas supply

4. A tubular membrane oxygen gas supply device, comprising:
a) a tubular hydrated ion-exchange separator defining an inner surface, an outer surface and a port;
b) an outer catalytic membrane at the outer surface;
c) an inner catalytic membrane at the inner surface, wherein the inner catalytic membrane has an inner surface that defines, at least in part, an inner tubular volume that is in fluid communication with the port;
d) a manifold in fluid communication with the outlet, whereby application of an electromotive force across the outer and inner catalytic membranes will cause a cathodic reaction of an oxygen gas component of ambient air with hydrogen ions at one of either the outer catalytic membrane or the inner catalytic membrane to form water, and an anodic reaction of water at the other of the outer catalytic membrane and the inner catalytic membrane to form oxygen gas and hydrogen ions, thereby causing oxygen gas to collect either within the inner tubular volume and pass through the port to the manifold as an oxygen gas supply, or at the outer surface of the tubular hydrated ion exchange separator as the oxygen gas supply.

5. The tubular membrane oxygen gas supply device of claim 4 wherein the outer catalytic membrane is a cathode and the inner catalytic membrane is an anode, and wherein the oxygen gas collects within the inner tubular volume and passes through the port at the oxygen gas supply

6. The tubular oxygen gas supply device of claim 4, wherein the inner catalytic membrane is a cathode, and the outer catalytic membrane is an anode, and wherein oxygen gas collects at the outer catalytic membrane as the oxygen gas supply

7. The oxygen gas supply device of claim 4, wherein the surface area-to-volume ratio, as defined by an outer surface of the outer catalytic membrane relative to the inner tubular volume, is equal to or greater than about 16:1 in²/in³.

8. The oxygen gas supply device of claim 4, wherein the tubular hydrated ion-exchange membrane includes at least one material selected from the group consisting of perfluorsulfonic acid and polysulfone.

9. The oxygen gas supply device of claim 4, wherein the tubular hydrated ion-exchange membrane has a water content of 0-40%.

10. The oxygen gas supply device of claim 4, further including a direct current power source in selective electrical communication with the inner and outer catalytic membranes.

11. The oxygen gas supply device of claim 4, wherein the inner and outer catalytic membrane are each independently selected from the group consisting of platinum, iridium oxide and ruthenium oxide.

12. The oxygen gas supply device of claim 4, wherein the inner tubular volume has a diameter in a range of between about 0.010 inches and 0.499 inches.

13. The oxygen gas supply device of claim 4, wherein the tubular hydrated ion-exchange membrane has a thickness of between about 0.001 inches and about 0.020 inches.

14. The oxygen gas supply device of claim 4, wherein the inner and outer catalytic membranes each independently have a thickness in a range of between about 0.0001 inches and about 0.5000 inches.

15. The oxygen gas supply device of claim 4, wherein the tubular hydrated ion-exchange membrane has an axial length in a range of between about 0.125 inches and about 24.0 inches.

16. The oxygen gas supply device of claim 4, wherein the tubular hydrated ion-exchange membrane has a cross-sectional shape that is selected from the group consisting of cylindrical and polygonal.

17. The oxygen gas supply device of claim 15, wherein the cross-sectional shape of the tubular hydrated ion-exchange membrane is polygonal.

18. The oxygen gas supply device of claim 16, wherein the polygonal shape is selected from the group consisting of triangular, rectangular and square.

19. The oxygen gas supply device of claim 4, wherein the oxygen gas supply includes a plurality of tubular hydrated ion-exchange membranes, each of which includes corresponding inner and outer catalytic membranes, the inner tubular volumes each being defined by inner surfaces of the inner catalytic membranes and in fluid communication with the manifold, and further including terminals for electrical communication of the inner and outer catalytic membranes to a direct current power source.

20. The oxygen gas supply device of claim 19, further including a housing that encloses the tubular hydrated ion-exchange membranes and corresponding inner and outer catalytic membranes, the housing defining an opening that provides fluid communication between an external surface of the housing and the outer catalytic membranes.

21. The oxygen gas supply device of claim 20, further including a tube attached to the manifold and a regulator attached to the tube, whereby the supply of oxygen gas from the manifold to a patient is regulated.

22. The oxygen gas supply device of claim 21, wherein the electromotive force applied to the inner and outer cathodes is limited to no more than about 1.5 volts to suppress hydrogen gas formation and to enable sustained operation using the inherent hydration content of the ion-exchange membrane.

23. A method for separating oxygen gas from air, comprising the steps of:
a) exposing a tubular membrane to ambient air, the tubular membrane including an outer catalytic membrane, an inner catalytic membrane within the outer catalytic membrane, and a tubular hydrated ion-exchange membrane contacting and partitioning the outer and inner catalytic membranes, the tubular membrane including a port in fluid communication with an inner tubular volume defined, at least in part, by an inner surface of the inner catalytic membrane;
b) applying an electromotive force between the outer and inner catalytic membranes, whereby an oxygen gas component of the ambient air will react in a cathodic reaction at one of the outer catalytic membrane and the inner catalytic membrane with hydrogen ions to form water, and water at the other of the outer catalytic membrane and the inner catalytic membrane will react in an anodic reaction to form oxygen gas and hydrogen ions, whereby the oxygen gas collects at either the inner catalytic membrane or the outer catalytic membrane; and
c) collecting the oxygen gas through the port or from the outer catalytic membrane.

24-43. (canceled)

44. A portable wearable oxygen gas supply device, comprising:
a) a housing defining a first port and a second port, and configured to be worn by a subject;
b) a plurality of tubular membranes within the housing, each of the tubular membranes including:
i) a tubular hydrated ion-exchange membrane defining an inner surface, and an outer surface,
ii) an outer catalytic membrane at the outer surface in fluid communication with the first port, and
iii) an inner catalytic membrane at the inner surface wherein the inner catalytic membrane has an inner surface that defines an inner tubular volume in fluid communication with the second port;
c) terminals for connection of the outer and inner catalytic membranes to a direct current power source;
d) a manifold in fluid communication with the second port, whereby application of an electromotive force across the tubular hydrated ion-exchange membrane by direct current from a power source will cause a catalytic reaction of an oxygen component of ambient air at one of the outer catalytic membranes and the inner catalytic membranes with hydrogen ions to form water and an anodic reaction of water at the other of the outer catalytic membranes and the inner catalytic membranes to form oxygen gas and hydrogen ions, thereby causing oxygen gas to collect either within the inner tubular volumes and pass through the second port or at the outer catalytic membrane and pass through the first port to the manifold separate from the ambient air to thereby form a supply of oxygen gas;
e) a conduit extending from either the first port or the second port the housing, whereby the subject can access the supply of oxygen gas.

45-60. (canceled)
